(19) United States
(12) Patent Application Publication
(10) Pub. No.: US 2011/0281834 A1
(43) Pub. Date: Nov. 17, 2011

(54) Pharmaceutical Formulations for Iontophoretic Delivery of a Corticosteroid

(76) Inventors: Phillip M. Friden, Bedford, MA (US); Hyun Kim, Weston, MA (US); Bireswar Chakraborty, Andover, MA (US)

(21) Appl. No.: 13/128,579

(22) PCT Filed: Nov. 10, 2009
(86) PCT No.: PCT/US2009/063820

§ 371 Date: Jul. 29, 2011

Related U.S. Application Data
(60) Provisional application No. 61/112,997, filed on Nov. 10, 2008.

(51) Int. Cl. A61K 31/573; A61K 31/57; A61P 29/00; A61K 31/56; A61K 31/58

(52) U.S. Cl. 514/174; 514/179; 514/180

(57) ABSTRACT

The present invention provides pharmaceutical formulations suitable for iontophoresis that provide enhanced iontophoretic delivery of a corticosteroid to the skin.

### BACKGROUND OF THE INVENTION

An iontophoretic delivery system is, for example, a drug delivery system that releases drug at a controlled rate to the target tissue upon application. The advantages of systems wherein drug is delivered locally via iontophoresis are the ease of use, relatively safe administration, the ability to finely modulate the dose by changing the time of application and/or the current level and the ability to interrupt administration by simply stopping the current and/or peeling off or removing it from the skin or other body surface whenever an overdosing is suspected. The total skin surface area of an adult is about 2 m². In recent years, iontophoretic delivery of drugs has attracted wide attention as a better way of administering drugs for local as well as systemic effects. The design of iontophoretic delivery systems can usually be such that the side effects generally seen with the systemic administration of conventional dosage forms are minimized.

Iontophoresis has been employed for many years as a means for applying medication locally through a patient's skin and for delivering medicaments to the eyes and ears. The application of an electric field to the skin is known to greatly enhance the ability of the drugs to penetrate the target tissue. The use of iontophoretic transdermal delivery techniques has obviated the need for hypodermic injection for some medicaments, thereby eliminating the concomitant problems of trauma, pain and risk of infection to the patient.

Iontophoresis involves the application of an electromotive force to drive or repel ions through the dermal layers into a target tissue. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode.

U.S. Pat. No. 6,477,410 issued to Henley et al. describes the use of iontophoresis for drug delivery. However, there remains a need for improved formulations that facilitate the delivery of specific active agents such as corticosteroids. Topical anti-inflammatory glucocorticoids can be used in the treatment of various inflammatory conditions, including inflammatory skin conditions. The anti-inflammatory activity of these glucocorticoids is dependent on their penetration into the stratum corneum and partitioning into the epidermis and dermis where steroid receptors are localized. It would be advantageous to develop formulations of corticosteroids and methods of administering corticosteroids using iontophoresis that result in increased permeation of topically administered corticosteroids into the epidermis and dermis, that permit administration without pain and with minimal side effects, that allow the drug to be administered at lower doses and with less frequency and that result in increased residence time within the dermis and/or epidermis. The present invention fulfills these needs.

### SUMMARY OF THE INVENTION

The present invention provides pharmaceutical formulations suitable for iontophoresis that provide enhanced iontophoretic delivery of a corticosteroid to the skin. In another embodiment, the invention is directed to a method for administering a corticosteroid to a patient in need thereof comprising iontophoretically administering to a body surface of the patient a formulation comprising the corticosteroid. In yet another embodiment, the invention is directed to a method of treating an inflammatory condition comprising iontophoretically administering to a body surface of the patient a formulation comprising a corticosteroid.

In one embodiment, the invention is a formulation suitable for iontophoretic delivery of a corticosteroid comprising the corticosteroid in an amount from about 0.01 to about 30% (w/w), and a buffer system sufficient to maintain the pH of the formulation from about 4.0 and about 8.0. In another embodiment, the formulation further comprises a stabilizer.

In another embodiment, the invention is a formulation suitable for iontophoretic delivery of a glucocorticoid comprising the glucocorticoid in an amount from about 0.01 to about 30% (w/w), and a buffer system sufficient to maintain the pH of the formulation from about 4.0 and about 8.0. In another embodiment, the formulation further comprises a stabilizer.

In yet another embodiment, the invention is a formulation suitable for iontophoretic delivery of dexamethasone or a pharmaceutically acceptable derivative thereof in an amount from about 1 to about 30% (w/w) and a buffer system sufficient to maintain the pH of the formulation from about 5.0 and 7.5. In an additional embodiment, the formulation further comprises a stabilizer. In another embodiment, the formulation comprises a pharmaceutically acceptable derivative of dexamethasone wherein the derivative is dexamethasone sodium phosphate.

In a further embodiment, the invention is a formulation suitable for iontophoretic delivery of triamcinolone or a pharmaceutically acceptable derivative thereof in an amount from about 0.1 and 1.0% (w/w), a buffer system sufficient to maintain the pH of the formulation from about 4.5 and about 7.0 and a solublizer. In another embodiment, the formulation further comprises a stabilizer. In an additional embodiment, the formulation comprises a pharmaceutically acceptable derivative of triamcinolone wherein the pharmaceutically acceptable derivative is triamcinolone acetonide.

In another embodiment, the invention is a method of administering a corticosteroid to a patient in need thereof comprising iontophoretically delivering a formulation comprising a corticosteroid to a body surface of said patient. In another embodiment, the corticosteroid is a glucocorticoid. In a further embodiment, the glucocorticoid is selected from the group consisting of dexamethasone, triamcinolone and pharmaceutically acceptable derivatives thereof.

In another aspect, the invention is a method of treating an inflammatory condition in a patient in need thereof comprising iontophoretically administering to the body surface of said patient a formulation comprising a corticosteroid to said patient. In a further aspect, the corticosteroid is a glucocorticoid. In a further embodiment, the glucocorticoid is selected from the group consisting of dexamethasone, triamcinolone and pharmaceutically acceptable, derivatives thereof. In one embodiment, the inflammatory condition is an inflammatory skin condition. In another embodiment, the inflammatory skin condition is acne.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings. The drawings are not necessarily to scale, emphasis instead being placed upon illustrating the principles of the invention. In the drawings:

FIG. 1 depicts the results of an example experiment assessing the cumulative permeability (ug/cm²) over time (hours) of dexamethasone sodium phosphate through full thickness hairless rat skin using either cathodal or anodal iontophoresis.

FIG. 2 depicts the results of an example experiment assessing the cumulative permeability (ug/cm²) over time (hours) of dexamethasone sodium phosphate through full thickness hairless rat skin after 0.4 mA/cm² iontophoresis for 1 hour.

FIG. 3 depicts the results of an example experiment assessing the cumulative permeability (ug/cm²) over time (hours) of dexamethasone sodium phosphate through full thickness hairless rat skin after 0.4 mA/cm² iontophoresis for 30 minutes, 1 hour, 4 hour or by passive delivery.

FIG. 4 depicts the results of an example experiment assessing the cumulative permeability (ug/cm²) over time (hours) of 10, 25 and 50 mg/ml concentrations of dexamethasone sodium phosphate through full thickness hairless rat skin after 0.2 mA/cm² cathodal iontophoresis for 1 hour.

FIG. 5 depicts the results of an example experiment assessing the concentration of dexamethasone sodium phosphate in tissue (ug/ml) over time (hours) after 0.2 mA/cm² cathodal iontophoresis for 1 hour.

FIG. 6A depicts the results of an example experiment assessing the amount of dexamethasone sodium phosphate in the stratum corneum, underlying skin or both after 0.2 mA/cm² cathodal iontophoresis for 1 hour or after passive delivery.

FIG. 6B depicts the results of an example experiment assessing the amount of dexamethasone sodium phosphate in the stratum corneum, underlying skin or both after 0.2 mA/cm² cathodal iontophoresis for 15 minutes or after passive delivery.

FIG. 6C depicts the results of an example experiment assessing the amount of dexamethasone sodium phosphate in the stratum corneum, underlying skin or both after 0.2 mA/cm² cathodal iontophoresis for 15 minutes or after passive delivery measured 0, 24 and 48 hours after iontophoretic administration.

FIG. 7A depicts a series of confocal microscopy images taken from the surface of hairless rat skin to a depth of 50 micrometers (um) after anodal iontophoresis of DEX-fluorescein at 0.2 mA/cm² for 15 minutes.

FIG. 7B depicts a series of confocal microscopy images taken from the surface of hairless rat skin to a depth of 50 micrometers (um) after passive treatment of DEX-fluorescein.

### DETAILED DESCRIPTION OF THE INVENTION

The present invention relates generally to pharmaceutical formulations suitable for iontophoresis that provide enhanced iontophoretic delivery of a corticosteroid to the skin. In one embodiment, the invention is directed to a method for administering a corticosteroid to a patient in need thereof comprising iontophoretically administering to a body surface of the patient a formulation comprising the corticosteroid. In another embodiment, the invention is directed to a method of treating an inflammatory condition comprising iontophoretically administering to a body surface of the patient a formulation comprising a corticosteroid.

In various embodiments, the formulation conditions maintain the drug in a mostly ionized state at a high concentration and is non-irritating. In various embodiments, the theological property of the formulation is compatible and adequate to incorporate the formulation in an iontophoretic applicator for loading and retention. Preferably, the formulation is stable under storage condition as well as during iontophoresis.

In one embodiment, the invention is a formulation suitable for iontophoresis comprising a corticosteroid. In another embodiment, the formulation comprises a corticosteroid, a stabilizer and a buffer system, wherein the buffer system is capable of maintaining the pH of the formulation from about 4 to about 8. In another embodiment, the corticosteroid is present in an amount from about 0.01 to about 30% (w/w).

In another embodiment, the inventive formulation comprises one or more agents selected from the group consisting of an agent that has the ability to slow the release of the corticosteroid from the epidermis to the dermis, a preservative, a thickening agent, an emollient and a solubilizing agent.

In other various embodiments, the inventive formulation further comprises a stabilizing agent. Stabilizers (or “stabilizing agents”) include, for example, alcohols, antioxidants and chelating agents and combinations thereof. Exemplary alcohols are benzyl alcohol and ethanol. Exemplary chelating agents included EDTA and disodium EDTA. Antioxidants include, for example, butylated hydroxy toluene, butylated hydroxy anisole, TPGS, sodium sulfite, ascorbic acid, vitamin E, creatine and methionine.

Buffer systems include those buffers and combinations of buffers that maintain the pH of the formulation from about 4 to about 8. Examples of suitable buffers include phosphate buffer, citrate buffer, acetate buffer, piperazine-N,N′-bis(2-ethanesulfonic acid) (PIPES) buffer, dimethyl arsenate (Cacodylate) buffer and 2-(N-morpholino)ethanesulfonic acid (MES) buffer, and combinations thereof. In one embodiment, the buffer system is selected from the group consisting of citrate buffer, a phosphate buffer and a combination thereof.

An agent that slows release of the corticosteroid from the epidermis to the dermis is an agent that has the ability to increase the residence time of the corticosteroid and/or creates a depot effect to maximize efficacy and minimize skin atrophy. Exemplary agents of this category include saturated and unsaturated fatty acids, polyethylene glycol, glycol ethers and combinations thereof. As will be appreciated by one having skill in the art, polyethylene glycol (PEG) of various molecular weights can be used in the inventive formulation, including, but not limited to polyethylene glycol PEG 200, PEG 400, PEG 600, PEG 1000 and PEG 3350. Exemplary saturated fatty acids include stearic acid and isostearic acid. Exemplary unsaturated fatty acids include oleic acid and linoleic acid.

In other various embodiments, the inventive formulation further comprises a preservative. Preservatives include, but are not limited to, sodium benzoate, benzalkonium chloride, parabens (including methyl and propyl paraben), and combinations thereof. In one embodiment, the preservative is benzalkonium chloride.

In some embodiments, the inventive formulation further comprises a thickening agent. A thickening agent is an agent that is capable of modulating the viscosity or thickness of the formulation. Such thickening agents include but are not limited to, ionic and non-ionic, high viscosity, water soluble polymers; crosslinked acrylic acid polymers such as the “carbomer” family of polymers, e.g., carboxypolyalkylenes that can be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose (HEC), hydroxypropyl methylcellulose, hydroxypropyl methylcellulose phthalate, methyl cellulose, carboxymethyl cellulose, and etherified cellulose; gums such as tragacanth and xanthan gum; sodium alginate, calcium alginate; gelatin, hyaluronic acid and salts thereof, chitosans, gellans, poloxamers, polyacrylates, or any combination thereof. In one embodiment, the thickening agent is selected from the group consisting of hydroxyethyl cellulose, polyvinylpyrrolidone and a combination thereof.

In various embodiments, the inventive formulation comprises an emollient. An emollient is a material capable of preventing or relieving dryness, as well as for the protection of the skin. A wide variety of suitable emollients is known in the art and may be used herein. One example of an emollient is glycerin.

In some embodiments, the inventive formulation comprises an solubilizer. A solubilizer is an agent that enhances the solubility of the corticosteroid in a solution. Such agents are well-known in the art. Solubility enhancers suitable for use in the present invention include, but are not limited to, polyethylene glycol, propylene glycol, polysorbate, Cremophor (poloxyethylated castor oil) and combinations thereof.

In one embodiment, the formulation suitable for iontophoresis comprises dexamethasone or a pharmaceutically acceptable derivative thereof in an amount from about 0.01 and about 30% (w/w). In another embodiment, the formulation comprises dexamethasone or a pharmaceutically acceptable derivative thereof in an amount of about 0.1 and about 25%. In another embodiment, the formulation comprises dexamethasone or a pharmaceutically acceptable derivative thereof in an amount from about 1.0 and about 20% (w/w). In a further embodiment, the formulation comprises dexamethasone or a pharmaceutically acceptable derivative thereof in an amount from about 5 and about 20% (w/w).

In another embodiment, the formulation comprises dexamethasone or a pharmaceutically acceptable derivative thereof and a buffering system capable of maintaining the pH of the formulation from about 5.0 and about 7.5. In yet another embodiment, the formulation further comprises a stabilizer. In certain embodiments, the dexamethasone pharmaceutically acceptable derivative is dexamethasone sodium phosphate. In one embodiment, the dexamethasone sodium phosphate is included in the formulation at a concentration of about 20 to about 300 mg/ml. In a further embodiment, the dexamethasone sodium phosphate is included in the formulation at a concentration of about 20 and about 200 mg/ml. In one embodiment, the dexamethasone sodium phosphate is included in the formulation at a concentration of about 25 mg/ml. In another embodiment, the dexamethasone sodium phosphate is included in the formulation at a concentration of about 150 mg/ml.

In one aspect embodiment, the formulation comprises a buffer system selected from the group consisting of a citrate buffer, a phosphate buffer and a combination thereof. In another embodiment, the formulation has a pH of about 7.2 to about 7.6. In another embodiment, the formulation comprises dexamethasone sodium phosphate, a phosphate buffer and has a pH of about 7.2 to about 7.6.

In certain embodiments, the inventive formulation comprising dexamethasone or a pharmaceutically acceptable derivative thereof further comprises one or more agents selected from the group consisting of a stabilizer, an agent that has the ability to slow the release of the corticosteroid from the epidermis to the dermis, a preservative, a thickening agent, an emollient and a solubilizing agent.

In another aspect, the formulation comprises dexamethasone sodium phosphate in an amount from about 1 to about 30% (w/w), has a pH from about 5.0 and about 7.5 and comprises one or more additional components listed in Table A, in the following amounts:

TABLE A

| Component | Composition % (w/w) |
|---|---|
| Alcohol | About 1 to about 10 |
| Chelator | About 0.01 to about 0.1 |
| Antioxidant | About 0.01 to 0.1 |
| Phosphate buffer, Citrate buffer or | About 0.1 to about 1 |
| combination thereof | |
| Agent that slows release of drug from | About 0.1 to about 50 |
| dermis to epidermis | |
| Preservative | About 0.01 to about 0.1 |
| Thickening Agent | About 0.1 to about 10 |
| Emollient | About 1 to about 30 |

In another aspect, the formulation comprises dexamethasone sodium phosphate in an amount from about 1 to about 30% (w/w), phosphate buffer and one or more additional components listed in Table B in the following amounts, wherein the formulation has a pH from about 7.2 to about 7.6:

TABLE B

| Component | Composition % (w/w) |
|---|---|
| Benzyl alcohol and/or ethanol | About 1 to about 15 |
| EDTA and/or disodium EDTA | About 0.01 to about 0.1 |
| Butylated hydroxy anisole and/or | About 0.01 to about 0.1 |
| butylated hydroxy toluene | |
| Benzalkonium chloride | About 0.01 to about 0.02 |
| Hydroxyethyl cellulose and/or polyvinyl | About 0.1 to about 10 |
| pyrrolidone | |
| Glycerin | About 1 to about 30 |

It is to be understood that the formulation can comprise one or more of the components described in the tables in the present application. In addition, it is to be understood that the formulation can comprise one or more of each type of component described in the tables herein and each component can be included in the formulation in the indicated amount; for example, the formulation may comprise one or more stabilizers and each stabilizer can be included in the formulation in an amount (from about 1 and about 10% (w/w)).

In certain other aspects, the formulation suitable for iontophoresis comprises triamcinolone or a pharmaceutically acceptable derivative thereof in an amount from about 0.001 to about 3.0% (w/w). In another embodiment, the formulation comprises triamcinolone or a pharmaceutically acceptable derivative thereof in an amount from about 0.01 to about 1.0% (w/w). In one embodiment, the formulation comprises triamcinolone or a pharmaceutically acceptable derivative thereof and a buffer system sufficient to maintain the pH of the formulation from about 4.5 and 7.0.

In another embodiment, inventive formulation comprising triamcinolone or a pharmaceutically acceptable derivative thereof further comprises a stabilizer. In a further embodiment, the solubilizing agent is selected from the group consisting of polyethylene glycol, propylene glycol, polysorbate, Cremophor and combinations thereof. In certain embodiments, the stabilizer is selected from the group consisting of Cremophor, an alcohol, polyethylene glycol and combinations thereof. In certain other embodiments, the formulation comprises about 15% Cremophor, 25% ethanol and 25% PEG 400.

In a further embodiment, the inventive formulation comprising triamcinolone or a pharmaceutically acceptable derivative thereof further comprises one or more agents selected from the group consisting of an agent that has the ability to slow the release of the corticosteroid from the epidermis to the dermis, a preservative, a thickening agent, and an emollient.

In another aspect, the formulation comprises triamcinolone acetonide in an amount from about 0.01 to about 1.0% (w/w) and one or more additional components listed in Table C in the following amounts:

TABLE C

| Component | Composition % (w/w) |
|---|---|
| Alcoholic stabilizer | About 1 to about 25 |
| Chelator | About 0.01 to about 0.1 |
| Antioxidant | About 0.01 to about 0.1 |
| Buffer system sufficient to control pH of | About 0.1 to about 1.0 |
| formulation from about 4.5 and 7.0 | |
| Solubilizing agent | About 10 to about 40 |
| Agent that slows release of drug from | About 0.1 to about 50 |
| dermis to epidermis | |
| Preservative | About 0.01 to about 0.02 |
| Thickening Agent | About 0.1 to about 10 |
| Emollient | About 1 to about 30 |

In certain aspects the triamcinolone pharmaceutically acceptable derivative thereof is triamcinolone acetonide. In one aspect, the formulation comprises triamcinolone acetonide in an amount from about 0.01 to about 1.0% (w/w), a buffer system sufficient to control the pH of the formulation from about 4.5 and about 7.0 and a solubilizing agent. In another embodiment, the buffer system is a citrate buffer, a phosphate buffer or a combination thereof. In various embodiments, the inventive formulation comprising triamcinolone acetonide further comprises a stabilizer. In one embodiment, the stabilizer is selected from the group consisting of an alcohol, a chelator, an antioxidant and combinations thereof.

In an additional embodiment, the formulation comprising triamcinolone acetonide in an amount from about 0.01 to about 1.0% (w/w), a citrate buffer, about 15% Cremophor, 25% ethanol and 25% PEG 400, wherein the formulation has a pH of about 5.0. In one embodiment, this formulation further comprises a stabilizer.

In a further embodiment, the invention relates to a method of administering a corticosteroid to a patient in need thereof comprising iontophoretically administering to a body surface of the patient a corticosteroid. In another embodiment, the invention is directed to a method of administering a corticosteroid to a patient in need thereof comprising iontophoretically administering to the body surface of the patient a formulation of the invention. In various embodiments, the corticosteroid is a glucocorticoid. In some embodiments, the glucocorticoid is selected from the group consisting of dexamethasone, triamcinolone and pharmaceutically acceptable derivatives thereof.

Exemplary body surfaces to which the inventive method of administration and treatment are directed include, for example, the skin, the nails and the eyes.

In one embodiment, a current density sufficient for permeation of the formulation into the body surface is applied. In a further embodiment, a current density of at least about 0.01 mA/cm² is applied. In another embodiment, a current density of at least about 0.1 mA/cm² is applied. In yet another embodiment, a current density of at least about 0.2 mA/cm² is applied. In a further embodiment, a current density of about least about 0.4 mA/cm² is applied.

The iontophoresis can be applied for a sufficient time to achieve an effective amount of permeation. For example, a sufficient time for application is a time from about 1 minute to about 4 hours. In one embodiment, iontophoresis is applied for a time from about 15 minutes to about 2 hours. In another embodiment, iontophoresis is applied for a time of about 10 minutes.

In another embodiment, the corticosteroid is iontophoretically administered to the body surface at least twice. In a further embodiment, the corticosteroid can be iontophoretically administered to the body surface at least three times. In a further embodiment, the corticosteroid is iontophoretically administered to the body surface at least one time per week. In another embodiment, the corticosteroid is iontophoretically administered at an interval from once a week to once every four weeks.

In one aspect, the invention is a method of administering dexamethasone sodium phosphate to a body surface comprising cathodal iontophoresis of anionic dexamethasone sodium phosphate to said body surface.

In one embodiment, the inventive formulation comprising a corticosteroid is administered using an iontophoretic delivery device. In another embodiment, the formulation is laminated or allowed to soak into a foam material or non-woven medicinal grade fabric and applied to the body surface. In yet another embodiment, the formulation is preloaded into the applicator and distributed as a single use, single dose applicator for administration using an iontophoretic delivery device. Examples of iontophoretic delivery devices useful with the compositions and methods of the invention include, but are not limited to, handheld devices and devices which comprise a separate compartment as a power supply. Exemplary devices include, but are not limited to, those described in U.S. Pat. Nos. 6,148,231, 6,385,487, 6,477,410, 6,553,253, and U.S. Patent Publication Numbers 2004/0111051, 2003/0199808, 2004/0039328, 2002/0161324, and U.S. Application Ser. No. 60/743,528, all incorporated herein by reference. An example of an applicator which can be used with a formulation of the invention comprises an active electrode adhered to an open cell polymer foam or hydrogel. Another applicator which has been developed for use with a device for iontophoretic delivery of an agent to a treatment site comprises an applicator head having opposite faces and including an active electrode and a porous pad (such as a woven or non-woven polymer, for example, a polypropylene pad); a margin of the applicator head about the active electrode having a plurality of spaced projections there along; the porous pad and the applicator head being ultrasonically welded to one another about the margin of the head with the electrode underlying the porous pad; and a medicament or a medicament and an electrically conductive carrier therefor carried by the porous pad in electrical contact with the electrode.

In another embodiment, the invention is a method of treating an inflammatory condition in a patient suffering therefrom comprising iontophoretically administering a formulation of the invention to a body surface of the patient. In one embodiment, the inflammatory condition is an inflammatory skin condition. In another embodiment, the inflammatory condition is an inflammatory eye condition.

In a certain embodiment, the inflammatory skin condition is selected from the group consisting of eczema and related conditions, insect bites, dermatitis, erythroderma, mycosis fungoides, Pyoderma gangrenosum, Erythema multiforme, rosacea, onychomycosis, urticaria, psoriasis and acne. As used herein, eczema related conditions include atopic eczema, acrodermatitis, contact allergic dermatitis, dyshydrotic eczema and seborrheic dermatitis. In another embodiment, the inflammatory skin condition is acne. In a further embodiment, the inflammatory skin condition is acne and the glueoeorticoid is dexamethasone, triamcinolone, or a pharmaceutically acceptable derivative thereof. In yet another embodiment, the patient is administered a formulation of the invention, wherein the formulation further comprises an agent that inhibits comedo formation and/or decreases sebum production.

In other embodiments, the inflammatory eye condition is selected from the group consisting of uveitis and macular degeneration. In a further embodiment, the inflammatory eye condition is treated by iontophretically administering an inventive formulation comprising triamcinolone or a pharmaceutically acceptable derivative thereof.

In a further embodiment, the method of treating an inflammatory condition comprises iontophoretically administering a formulation of the invention to a body surface of the patient and administering a second pharmaceutical agent effective in treating said inflammatory conditions. Exemplary second pharmaceutical agents include, for example, anti-inflammatory agents or antimicrobials. Exemplary anti-inflammatory agents include non-steroidal anti-inflammatory drugs (NSAIDs). Exemplary antimicrobials are tetracycline, doxycycline and clindamycin. An exemplary anesthetic is lidocaine. In one embodiment, the second pharmaceutical agent is applied topically to the body surface of the patient.

In yet another embodiment, the invention is a method of treating acne comprising iontophoretically administering a formulation of the invention to the skin of the patient and administering a second pharmaceutical agent effective in treating acne. Such second pharmaceutical agents effective in treating acne include, for example, benzoyl peroxide, alpha hydroxy acids and antibiotics. An exemplary antibiotic is clindamycin.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.3, 3, 4, 5, 5.7 and 6. This applies regardless of the breadth of the range.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. The invention is directed to pharmaceutical formulations suitable for iontophoresis that provide iontophoretic delivery of a corticosteroid to the skin, methods of administering a corticosteroid to a patient in need thereof comprising iontophoretically administering to a body surface of the patient a formulation comprising the corticosteroid and to methods of treating an inflammatory skin condition comprising iontophoretically administering to a body surface of the patient a formulation comprising a corticosteroid.

As used herein, the term “corticosteroid” is meant to encompass both naturally occurring and synthetic corticosteroids. Exemplary corticosteroids include both glucocorticoids and mineralocorticoids. Glucocorticoids, such as cortisol, control carbohydrate, fat and protein metabolism and are anti-inflammatory by preventing phospholipid release, decreasing eosinophil action and through a number of other mechanisms, Mineralocorticoids, such as aldosterone, control electrolyte and water levels, mainly by promoting sodium retention in the kidney. Corticosteroids that may be incorporated in the inventive formulation include, but are not limited to, alclometasone, aldosterone, beclomethasone, betamethasone, ciclesonide, clobetasol, cloprednol, cortisone, cortivazol, deoxycortisone, desonide, desoximetasone, dexamethasone, difluorocortolone, fiuclorolone, fludrocortisone, flumethasone, flunisolide, flucinolone, fluocinonide, fludroxycortide, fluocortin, fluocortisone, fluorocortolone, fluorometholone, flurandrenolone, fluticasone, halcinonide, hydrocortisone, icomethasone, meprednisone, methylpredinsone, mometasone, paramethasone, prednisolone, prednisone, rofleponide, RPR 106541, tixocortol, triamcinolone, hydrocortisone, triamcinolone, betamethasone, dexamethasone, clobetasol, fluticasone, mometasone, fludroxycortide, fluocinonide, alclometasone, difluorocortolone and fluoeinolone and their pharmaceutically active derivatives.

As used herein, the term “pharmaceutically active derivatives” expressly includes prodrugs and pharmaceutically acceptable salts. Pharmaceutically acceptable derivatives include, for example, dexamethasone sodium phosphate and soft steroids. Soft steroids have been described in the literature. Soft steroids are designed to be rapidly metabolized after performing their therapeutic function. An exemplary soft steroid is loteprednol etabonate.

A “therapeutically effective amount” is an amount which, alone or in combination with one or more other active agents, can control, decrease, inhibit, ameliorate, prevent or otherwise affect one or more symptoms of a disease or condition to be treated.

“Treating” or “treatment” includes the administration of the compositions, compounds or agents of aspects of the present invention to prevent or delay the onset of the symptoms, complications, or biochemical indicia of a disease, alleviating or ameliorating the symptoms or arresting or inhibiting further development of the disease, condition, or disorder.

As used herein, “emollient” refers to a material capable of preventing or relieving dryness, as well as for the protection of the skin. A wide variety of suitable emollients is known in the art and may be used herein. In one embodiment, the emollient is glycerin.

The term “solubilizer” refers to an agent that enhances the solubility of the corticosteroid in a solution. Such agents are well-known in the art. Solubility enhancers suitable for use in the present invention include, but are not limited to, polyethylene glycol, propylene glycol, polysorbate, Cremophor (poloxyethylated castor oil) and combinations thereof.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the formulations of the present invention and practice the claimed methods. The following working examples therefore, specifically point out the preferred embodiments of the present invention, and are not to be construed as limiting in any way the remainder of the disclosure.

### Example 1

### Method for Iontophoretic Drug Permeation Through Hairless Rat Skin

Full thickness abdominal skin freshly excised from sacrificed hairless rats was equilibrated in the receptor buffer and mounted on Franz diffusion cells (0.64 cm²). The donor compartment (0.5 ml) contained corticosteroid in an appropriate buffer. For dexamethasone sodium phosphate (DEX-P), a 25 mg/mL drug solution in phosphate buffer (50 mM, pH 7.4) was used for the donor compartment. For triamcinolone acetonide (TMCA), 3.5 mg/mL TMCA formulation (in 25% w/w PEG 400, 25% ETOH, 15% cremophor RH40, qs to 100% with citrate buffer pH 5.0) was used for the donor compartment. The receptor compartment for DEX-P contained phosphate buffer (pH 7.4) with 75 mM NaCl added to drive the electrochemistry. The receptor compartment for TMCA was citrate buffer with 20% ethanol. DEX-P (pKa 1.89, 6.4) is an anion at physiological pH and was therefore delivered by cathodal iontophoresis. TMCA has no charge at pH 5 and was therefore delivered by anodal iontophoresis to utilize electroosmosis. For DEX-P, the cathode (Silver-Silver Chloride) was placed in the donor chamber along with drug solution and the anode (Silver wire) was placed in the receptor chamber. For TMCA, the anode (Silver-Silver Chloride) was placed in the donor chamber along with drug solution and the cathode (Silver wire) was placed in the receptor chamber. Electric current (0.2 to 0.4 mA/cm²) was applied using a custom current control device for up to 1 hr, after which experiments were either terminated or allowed to continue passively for various durations. Samples (0.3 ml) were taken from the receptor chamber at predetermined time points following iontophoresis and were replaced with the same amount of receptor buffer. The apparatus was maintained at 37° C. with constant stirring in both the donor and receptor compartments to maintain sink conditions. The samples were then analyzed by HPLC to measure drug permeation through skin. For DEX-P, samples of receptor fluid (injection volume 20 uL, run time 8 min) were analyzed using a Waters Alliance HPLC with a reverse phase column (C₁₈, 250×3 mm I.D.; 5 μm) with a flow rate of 1.0 ml/min and UV detection at 254 nm. The mobile phase consisted of 75/25 ammonium acetate (5 mM) and acetonitrile. For TMCA, samples of receptor fluid (injection volume 20 uL, run time 8 min) were analyzed using the same HPLC system with a flow rate of 0.8 ml/min and UV detection at 237 nm. The mobile phase consisted of 70/30 methanol and water. The standard curve was linear over a range of 0.25-10 μg/ml with r²>0.999 for both drugs. Control passive experiments were conducted in the same way except for the application of electric current. Data were expressed as means±standard deviations. All experiments were performed in at least triplicate.

### Example 2

### Measurement of Corticosteroid Levels in the Stratum Corneum and Underlying Skin

Skin samples from in vivo studies were tape stripped to determine drug levels in the stratum corneum and the underlying skin extracted to determine drug levels in the lower epidermis and dermis using the following protocol. After wiping excess drug off the skin using a moist kimwipe, pre-weighed tape strips were placed on the area exposed to the drug and made sure they adhered to the skin by rolling with a glass rod for 30 seconds. After rolling, the tape strips were held from one end with a pair of forceps and ripped off the skin very quickly. The first strip was discarded to remove the remnants of the drug on the skin. This process was continued with 30 strips to fully remove the stratum corneum and each strip analyzed for drug content by immersing in 1 mL extraction buffer for 1 hr under gentle stirring (150 rpm). Phosphate buffer (pH 7.4) was used for DEX, while methanol was used for TMCA as extraction buffers. All tape strip extracts were analyzed with HPLC. To ensure that any potential base form of DEX was fully extracted from the tape strips, the strips after extraction with phosphate buffer were shaken with 1 mL of methylene chloride and 50 μL 0.1% HCl. However, no DEX base was detected, suggesting sufficient extraction with the phosphate buffer alone. TEWL values were also recorded after every five strips.

Methylene chloride and HCl was used for DEX as the extraction solvent. After tape stripping, the rats were euthanized and the underlying skin excised. Before proceeding for skin extraction studies, the weight of the excised skin piece was recorded. This excised skin was minced into small pieces and placed into glass vials. To this minced skin, 1 mL deionised water was added and shaken for 30 minutes on a shaker. To this 100 μL of 0.1% HCl was added and centrifuged. To the centrifuged sample 6 mL of methylene chloride was added and shaken for 30 minutes. The methylene chloride (organic) extract was then evaporated under nitrogen. The methylene chloride addition and evaporation step was repeated and combined with the previous extraction. After evaporation, the samples were reconstituted using 1 mL of acetonitrile, filtered and filled in HPLC vials and analyzed accordingly. For TMCA, 30/70 chloroform and methanol was used as the extraction solvent. Similar to DEX, the skin was minced and 10 mL extraction solvent was added and shaken overnight after which the organic phase was evaporated under nitrogen followed by reconstitution into 0.5 mL mobile phase before analysis by HPLC. Recovery efficiency of the drugs from the skin using the appropriate extraction methods was calculated to be 67.5% and 76.5% for DEX and TMCA, respectively, using known amounts of drugs. The measured drug levels from the skin were corrected for recovery.

### Example 3

### Determination of Polarity for Dexamethasone Sodium Phosphate Iontophoretic Delivery In Vitro

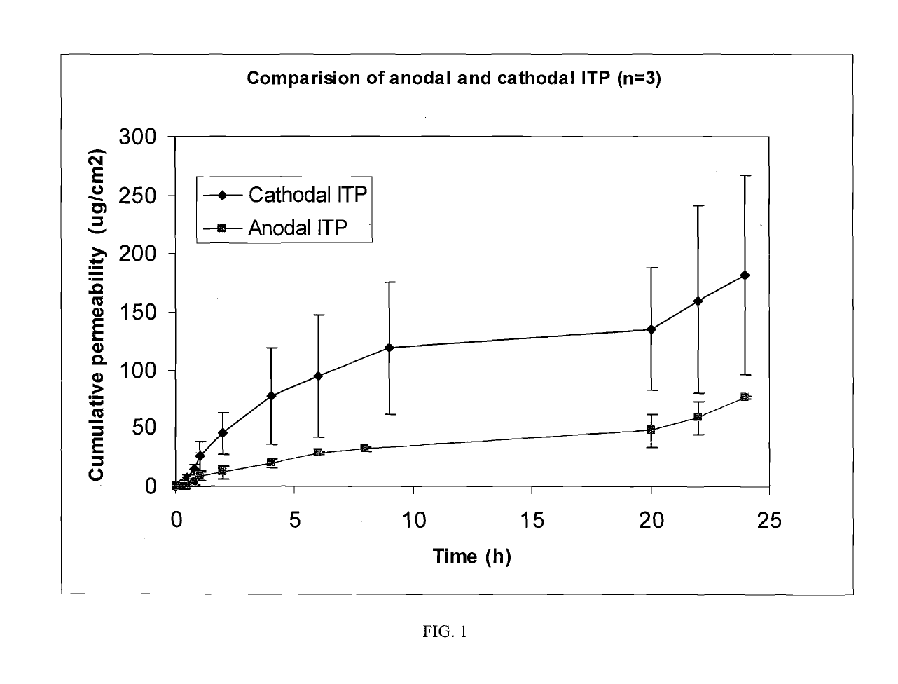

To determine whether anodal or cathodal iontophoresis resulted in improved skin permeation, DEX was evaluated by both anodal and cathodal delivery using the Franz diffusion cell fitted with full thickness hairless rat skin (formulation in contact with the stratum corneum layer on the skin). A formulation containing 25 mg/mL DEX in either a phosphate buffer (pH 7.4) or citrate buffer (pH 3.5) was used for cathodal and anodal delivery, respectively, at 0.4 mA/cm² for 2 hours and sampled for 24 hrs and plotted as a cumulative amount of DEX permeated with time. Cathodal iontophoresis run with the anionic DEX at neutral pH exhibited a significant improvement in permeation compared to anodal iontophoresis and was selected for further evaluation (FIG. 1).

### Example 4

### Effect of Current Application on the Permeation of Corticosteroids Through Skin In Vitro

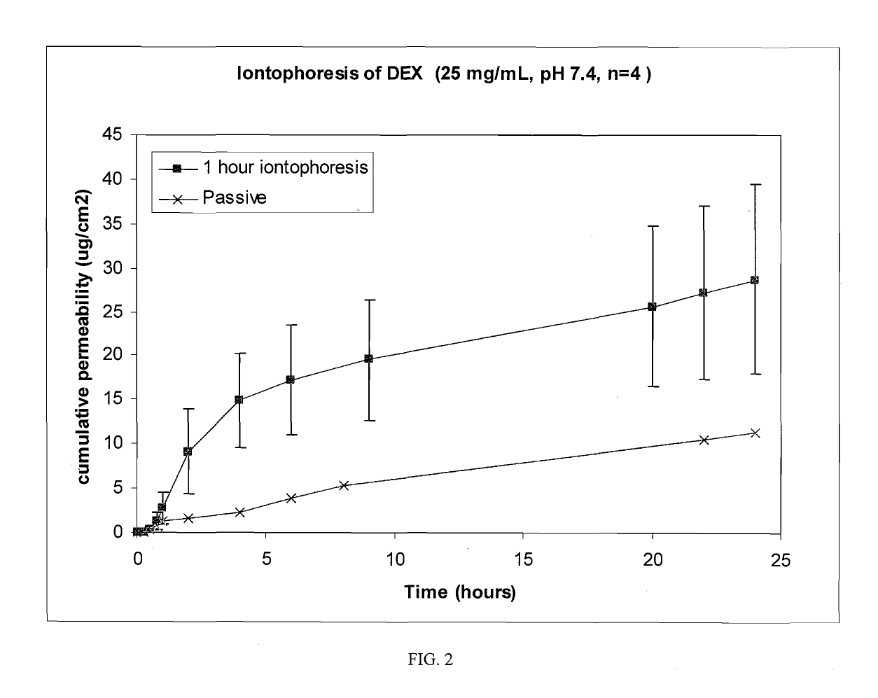

Cumulative DEX permeation through the hairless rat skin over 24 hrs was determined using 0.4 mA/cm² current density applied for 1 hr and sampled for 24 hrs in the Franz diffusion cell system. DEX concentration of 25 mg/mL in phosphate buffer (pH 7.4) was used in conjunction with cathodal iontophoresis and compared to passive delivery (no current). Compared to the initially low but gradual increase in passive permeation over 24 hrs, iontophoretic drug permeation increased quite rapidly during the initial several hours and continued to increase up to 24 hrs of study (FIG. 2). Application of current resulted in repulsion between the anionic DEX molecules and the cathode, which drove the drug ions through the skin.

### Example 5

### Effect of Current Duration on the Permeation of Dexamethasone Sodium Phosphate Through Hairless Rat Skin In Vitro

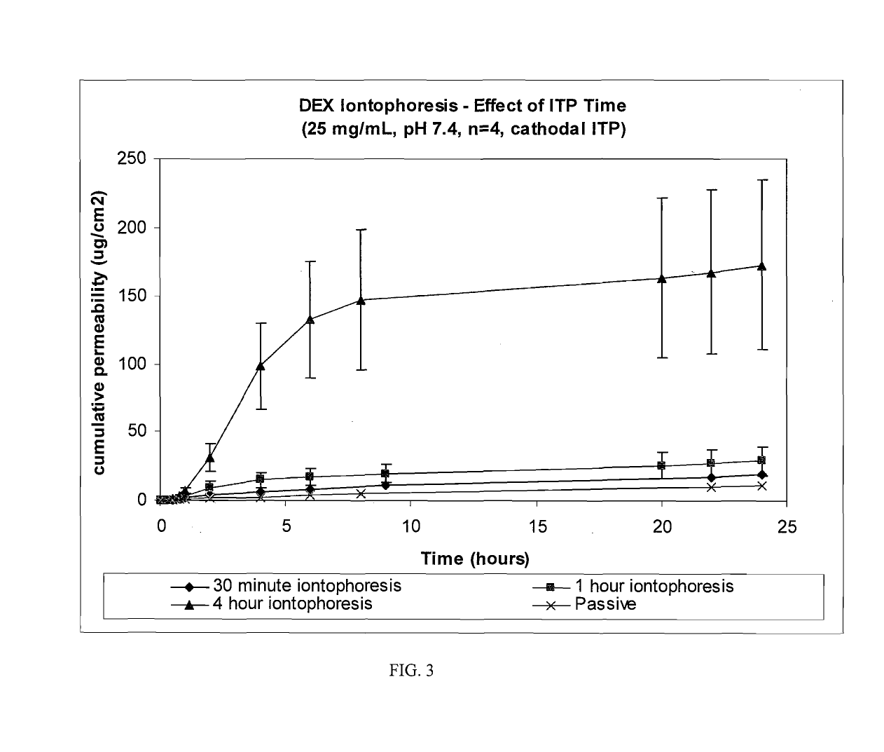

The effect of current duration (30 min, 1 hr, and 4 hr iontophoresis) on the permeation of 25 mg/mL DEX in phosphate buffer (pH 7.4) through hairless rat skin in vitro was evaluated at 0.4 mA/cm² current density using cathodal iontophoresis. Samples were removed periodically from the receptor and analyzed by HPLC. The results indicate that there is a current duration effect on the permeation of DEX (FIG. 3). All iontophoresis durations resulted in higher permeation over time compared to passive treatment, with the 4 hr iontophoresis treatment exhibiting the highest permeation.

### Example 6

### Effect of Dexamethasone Sodium Phosphate Concentration on Permeation Through Hairless Rat Skin In Vitro

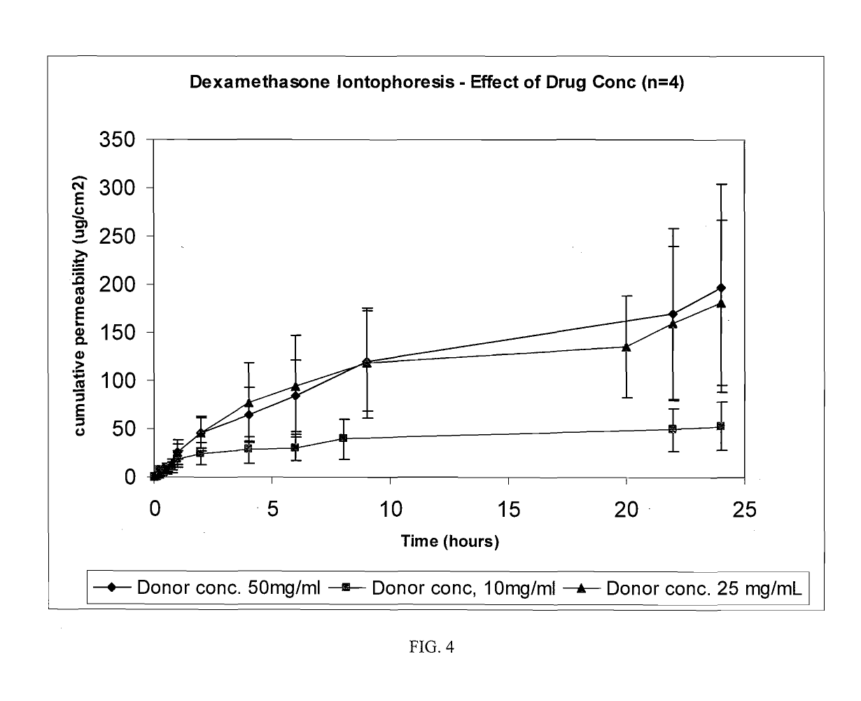

The effect of DEX concentration (10, 25, 50 mg/mL) on the permeation of DEX in phosphate buffer (pH 7.4) through hairless rat skin in vitro was evaluated at 0.2 mA/cm² current density for 1 hr using cathodal iontophoresis. Samples were measured periodically from the receptor and analyzed by HPLC over 24 hrs. The results indicate that there is a drug concentration effect on the permeation of DEX (FIG. 4). The lower DEX concentration of 10 mg/mL resulted in lower permeation over time compared to the higher drug concentrations (25 and 50 mg/mL) which were similar. As a result, 25 mg/mL DEX was selected for further evaluation.

### Example 7

### Microdialysis of Dexamethasone Sodium Phosphate in Hairless Rats in Vivo

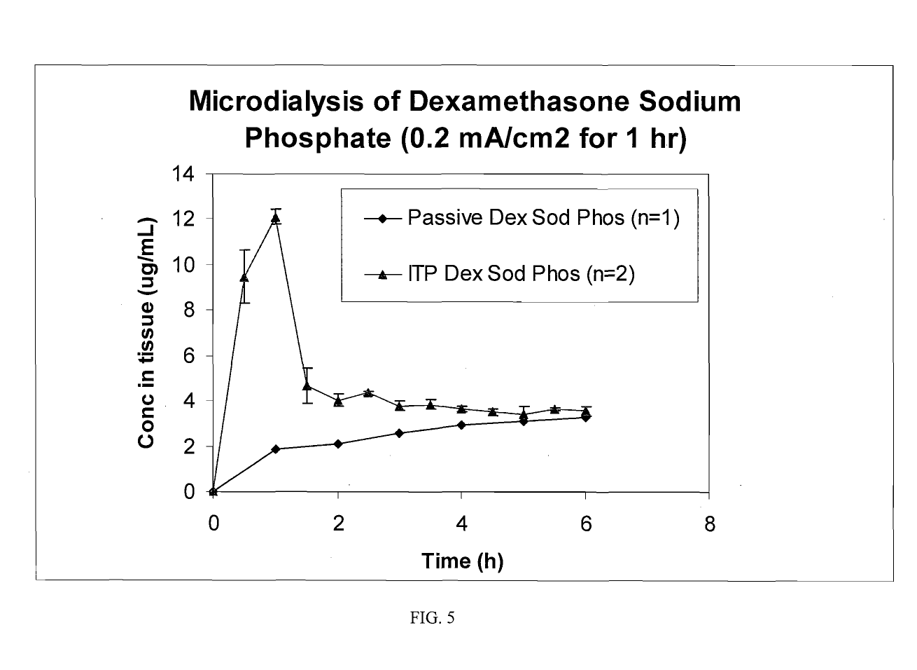

The aim of this work was to quantify the iontophoretic delivery of DEX to the dermal interstitial fluid. Experiments were conducted in vivo in a hairless rat model using microdialysis as a sampling technique. Hairless rats were anesthetized with ketamine (75 mg/kg) and xylazine (10 mg/kg) administered intraperitoneally. Once the rats were anaesthetized, the abdominal area was wiped with water and alcohol swabs. A linear probe was inserted intradermally into the skin exposed to the donor cartridge (stainless steel electrode; 3.14 cm²) preloaded with 250 uL of 25 mg/mL DEX in phosphate buffer (pH7.4). Cathodal iontophoresis (0.2 mA/cm² for 1 hr) was applied and the cartridge removed after the 1 hr of current. Samples were collected every 30 minutes for 6 hrs from the probe that was perfused with Lacated Ringer's at 0.7 uL/min and analyzed by HPLC. Recovery factor from retrodialysis was determined to be 0.19 and used as a correction factor for the drug concentration. As control, passive delivery of DEX was performed using the same conditions described except for the application of current (cartridge removed after 1 hr of passive delivery). In the iontophoresis treated rats, concentration of DEX in the interstitial fluid increased quickly during the 1 hr of iontophoresis and achieved a peak concentration of 12.10±0.34 ug/mL after 1 hr which decreased down to 3.57±0.19 ug/mL after 6 hrs, suggesting that iontophoresis can drive high levels of drug through the skin (FIG. 5). In contrast, DEX levels in the passive treated group were significantly lower but gradually increased during the course of 6 hrs.

### Example 8

### Triamcinolone Acetonide and Dexamethasone Sodium Phosphate Solubility

Triamcinolone acetonide is poorly ionized and has a poor solubility profile; to overcome this, individual as well as combinations of potential solubilizers were tested. TMCA (2.5 mg) was mixed into 1 mL of the individual components or mixtures of potential solubilizers and shaken overnight; drug was then added to all clear solutions which were then mixed overnight. The samples were then centrifuged and the supernatants analyzed by HPLC to determine drug solubility. Of the solubilizers evaluated, a combination of 15% cremophor, 25% ethanol, and 25% PEG 400 resulted in maximum solubility of 4.42 mg/mL (Table 1).

TABLE 1
Solubility of triamcinolone acetonide

| | Final conc |
|---|---|
| Solution (w/w) | (mg/mL) |
| 25% glycerin | 0.03 |
| 25% Propylene glycol | 0.06 |
| 25% ethanol | 0.23 |
| 25% PEG 400 | 0.07 |
| 25% PEG 3350 | 0.08 |
| 5% tween 80 | 0.12 |
| 15% cremophor | 0.48 |
| 15% cremophor, 25% ethanol | 1.38 |
| 15% cremophor, 25% ethanol, 25% PEG 400 | 4.42 |
| 15% cremophor, 25% ethanol, 25% PEG 3350 | 1.98 |
| 15% cremophor, 25% ethanol, 25% glycerin | 2.43 |
| 15% cremophor, 50% glycerin | 1.18 |
| 15% cremophor, 50% PEG 400 | 1.24 |

DEX-P (100 mg) was mixed into 1 mL of the individual components or mixtures of potential solubilizers and shaken overnight. Additional drug (100 mg) was added to clear solutions and mixed overnight. The samples were then centrifuged and the supernatants analyzed by HPLC to determine drug solubility.

TABLE 2
Solubility of dexamethasone sodium phosphate

| | Final conc |
|---|---|
| Solution (w/w) | (mg/mL) |
| Phosphate buffer | 70 |
| 15% ethanol (95%), 20% PEG400, 20% | 156 |
| glycerin, in phosphate buffer (w/w) | |
| 15% ethanol (95%), 30% PEG400, 20% | 197 |
| glycerin, in phosphate buffer (w/w) | |

### Example 9

### Topical and Transdermal Delivery of Triamcinolone Acetonide (TMCA) in Vitro and In Vivo

The effect of iontophoresis on the intradermal and transdermal permeation of TMCA using hairless rat skin in vitro was investigated using methods previously described. In addition, the amount of drug deposited in the stratum corneum and underlying skin after iontophoresis was quantified in vivo using tape stripping and skin extraction methods described previously. TMCA (3.5 mg/ml) was formulated in 15% cremophor RH40, 25% PEG 400 and 25% ethanol (95%), in citrate buffer pH 5.0. Anodal iontophoresis was performed for 1 h at 0.2 mA/cm². No detectable levels were observed in the receptor chamber for in vitro studies with TMCA, with or without iontophoresis. In the in vivo study, similar levels of TMCA were found in the stratum corneum after iontophoresis and passive delivery (16.43±1.99 μg and 19.66±1.35 μg, respectively). A significant difference between iontophoresis and passive delivery was observed in the amount of TMCA deposited in the underlying skin after tape stripping. Iontophoresis (0.2 mA/cm²) for 1 h delivered 1.15±0.32 μg when compared to passive delivery (0.34±0.08 μg). These results suggest that iontophoretic delivery of TMCA did not enhance the permeation of drug through the skin, indicating the possibility that a depot of TMCA is formed in the skin. The amount of TMCA deposited in the stratum corneum by iontophoresis was not significantly different from passive delivery. However, a significant difference was seen in the amount of TMCA deposited in underlying skin excised after tape stripping.

### Example 10

### In Vivo Skin Delivery of Dexamethasone Sodium Phosphate in Hairless Rats

The amount of dexamethasone sodium phosphate (DEX) delivered into the stratum corneum and the underlying skin (lower epidermis and dermis) in hairless rats following iontophoresis was determined using tape stripping and skin extraction methods, respectively, as described previously. A polypropylene non-woven cartridge (3.14 cm² area) with stainless steel electrode was filled with 300 uL of the mg/mL DEX formulation in phosphate buffer (pH 7.4). Hairless rats (n=4 per group) were anesthetized with ketamine (75 mg/kg) and xylazine (10 mg/kg) administered intraperitoneally. Once the rats were anaesthetized, the abdominal area was wiped with water and alcohol swabs. The cartridge loaded with the donor formulation was placed on the abdomen of each rat and connected to the power supply (Keithley Instruments, Cleveland, Ohio) for cathodal delivery. The counter electrode was placed around 3 cm away from the drug cartridge, and connected to the respective polarity of the power supply. The cartridge and the counter electrode were made sure to be in contact with the skin and a bandage was placed to prevent them from any further movement. Skin portion around the cartridge approximately 0.5 cm was marked for excision after the study to determine the lateral diffusion of the drug. Iontophoresis was conducted at a current density of 0.2 mA/cm² for 15 or 60 minutes. The donor cartridge was removed from the skin after iontophoresis and the skin was wiped with a moist kimwipe to remove any remaining drug on the skin. The skin was then tape stripped (30 strips) to remove the stratum corneum and the drug in the strips evaluated (with the first strip discarded for surface drug removal). The drug in the underlying skin was then extracted. TEWL (transepidermal water loss) base values were taken before and after tape stripping to confirm the removal of stratum corneum and this was confirmed. For passive studies the same procedure was followed without applying the current. In addition to evaluating drug levels immediately after the application of current (or passive), in some instances additional experiments to assess drug levels in the tape strips and skin at extended periods (e.g., 24, 48 hr . . . etc. after iontophoresis or passive) were performed to characterize the drug depot kinetics.

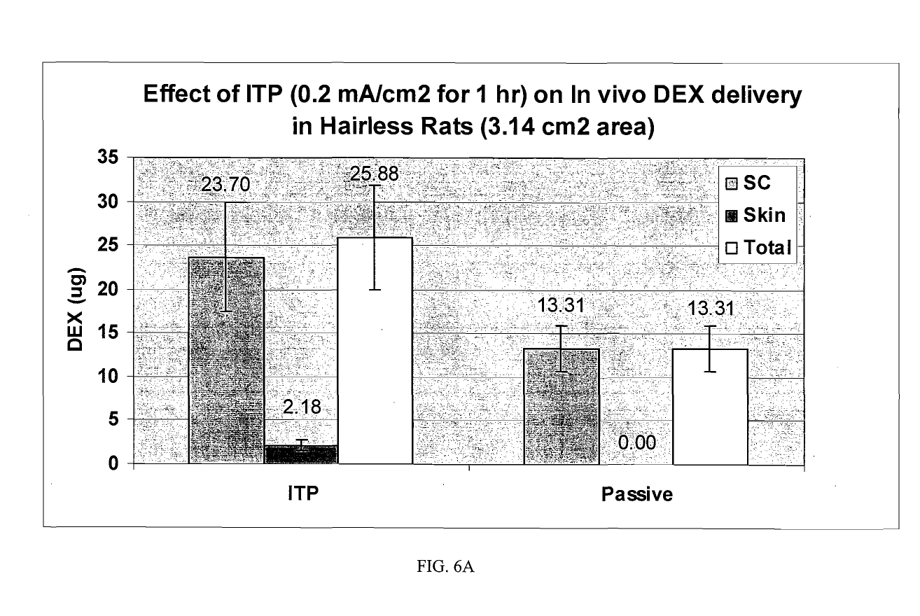

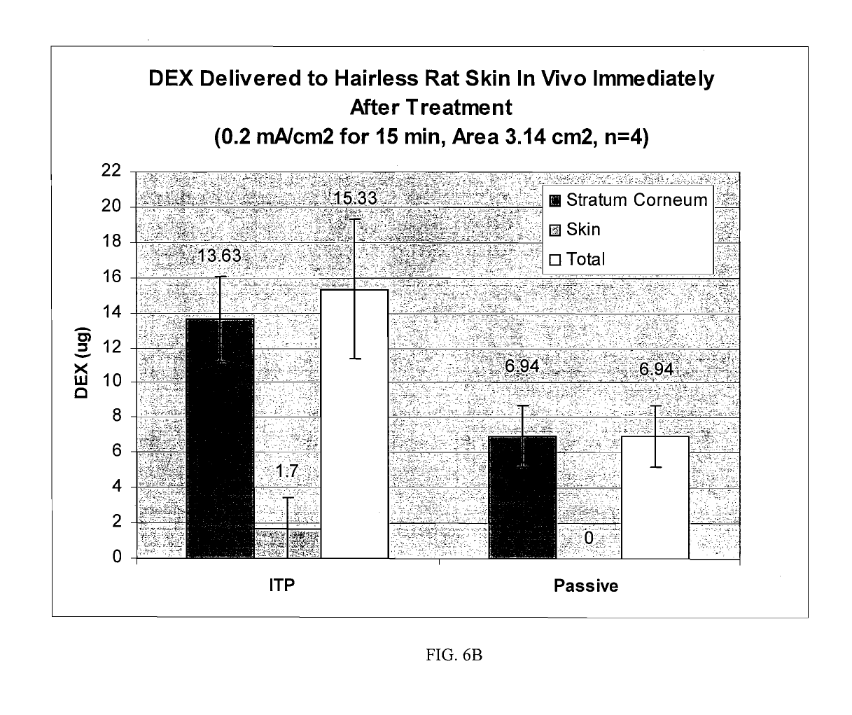

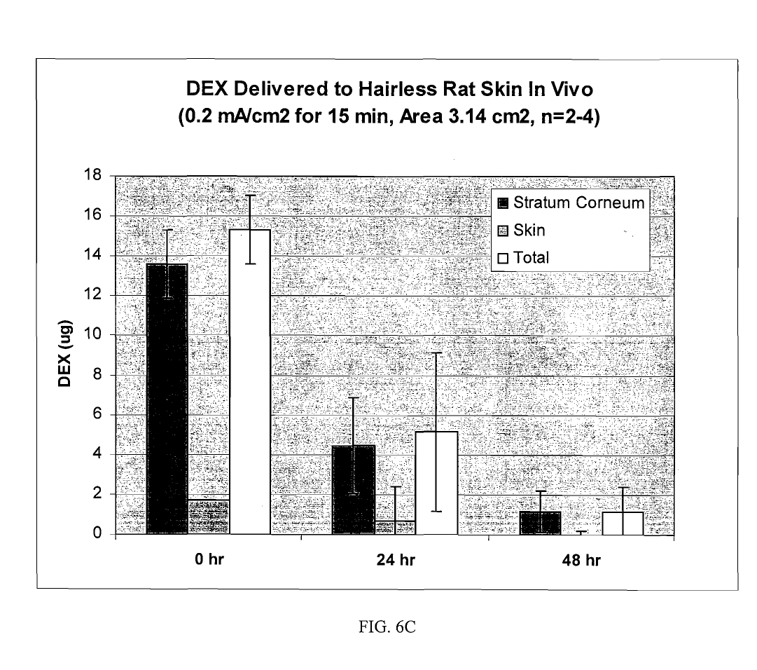

The in vivo skin delivery of DEX immediately following 60 minutes of iontophoresis was significant as compared to passive delivery (FIG. 6A). The total DEX delivered was 25.88±5.91 ug in the iontophoresis (ITP) treated group, compared to 13.31±2.69 ug for passive treatment. A fraction of the drug delivered was driven down into the underlying skin (2.18±0.69 ug) as compared to the stratum corneum (23.70±6.19 ug) in the ITP treated group, whereas all of the drug was localized in the stratum corneum layer for the passive group. These results indicate that iontophoresis may be driving the drug down deeper into the skin as well as in greater amounts. The drug concentration attained in the skin (stratum corneum and underlying skin) for the ITP treated group was 80.85±19.36 umol/kg which was projected to be greater than the estimated effective dose (0.2 umol/kg). No drug was detected in the peripheral skin areas outside the contact area of the foam, suggesting no lateral diffusion of DEX. Reducing the duration of iontophoresis to 15 minutes at the same current density (0.2 mA/cm2) decreased the initial drug levels in the stratum corneum (13.63±2.4 ug) and underlying skin (1.70±1.70 ug) moderately but still in excess of the estimated effective dose range (FIG. 6B). This initial level of drug in the skin decreased slowly over time and was detectable at 48 hrs after iontophoresis, suggesting some level of depot formation that retains and releases drug into the surrounding tissue over a period of time (FIG. 6C).

### Example 11

### Development of a Formulation Comprising Dexamethasone Sodium phosphate (DEX-P)

A stable, topical formulation suitable for iontophoretic delivery of steroids (glucocorticoids) and in particular, dexamethasone sodium phosphate is described herein. The formulation can be used for the treatment of acne. Topical treatment of acne focuses on reduction of inflammation, control of excess sebum production and unplugging of skin pores. For reduction of inflammation, an effective, moderately potent glucocorticoid like dexamethasone sodium phosphate salt is preferably used for iontophoretic delivery. The anti-inflammatory activity of the steroid depends on penetration into the stratum corneum and partitioning into the epidermis and dermis. The use of iontophoresis significantly enhances penetration of the steroid into epidermis and dermis where the steroid receptors are located. Availability of the steroid at the receptor, residence time and receptor affinity of the steroid determine the clinical efficacy. The vasoconstrictive activity of the steroids may reduce their local clearance. For iontophoretic delivery of steroid, several formulation criteria should be addressed. The formulation condition should maintain the drug in mostly ionized state at a high concentration and non-irritating. The rheological property of the formulation should be compatible and adequate to incorporate the formulation in the applicator for loading and retention. The formulation needs to be stable at storage condition as well as during iontophoresis. The formulation must meet antimicrobial effectiveness test. Based on the requirements for a stable, steroid formulation which meets the requirements of iontophoretic delivery, the formulation may include:
(1) a suitable anti-inflammatory glucocorticoid, for example, dexamethasone sodium phosphate in an amount (about 1 to about 30% w/w);
(2) a suitable stabilizer, for example, benzyl alcohol or ethanol, which also facilitates unplugging pores in an amount (about 1 to about 15%);
(3) a suitable stabilizer, chelator (preferably disodium edetate) and antioxidant such as butylated hydroxy anisole (preferred), creatine, sodium sulfite, or methionine;
(4) a suitable agent (preferably a saturated fatty acid and/or polyethylene glycol and/or glycol ether) which can increase residence time and build a depot effect (so that the steroid may be released from the epidermis to dennis very slowly to eliminate the possibility of skin atrophy);
(5) a suitable preservative, for example, benzalkonium chloride in an amount (about 0.01 to about 0.02%);
(6) a suitable buffer system, preferably citrate and/or phosphate sufficient to control pH from about 5.0 to about 7.5;
(7) a suitable thickener, for example, hydroxyethyl cellulose or polyvinyl pyrrolidone to build sufficient rheology of the formulation;
(8) an emollient, for example, glycerin in an amount about 1 to about 30%.

A stable, topical formulation suitable for iontophoretic delivery of steroids (glucocorticoids) and in particular, triamcinolone acetonide, for treatment of acne is described. The primary mode of delivery of this unionized drug would be by electroosmosis. Based on the requirements for a stable, aqueous steroid formulation which meets the requirements of iontophoretic delivery, the formulation may include:
(1) a suitable anti-inflammatory glucocorticoid, for example, triamcinolone acetonide in an amount (about 0.01 to about 1.0% w/w);
(2) a suitable stabilizer, preferably benzyl alcohol or ethanol, which also facilitates unplugging pores in an amount (about 1 to about 15%);
(3) a suitable stabilizer, chelator (for example, disodium edetate) and antioxidant such as butylated hydroxy anisole or butylated hydroxy toluene, creatine, sodium sulfite, or methionine;
(4) a suitable agent (for example, a saturated fatty acid and/or polyethylene glycol) which can increase residence time and build a depot effect (so that the steroid may be released from the epidermis to dermis very slowly to eliminate the possibility of skin atrophy);
(5) a suitable preservative, preferably benzalkonium chloride in an amount (about 0.01 to about 0.02%);
(6) a suitable solubilizing agent(s) preferably polyethylene glycol(s), propylene glycol, polysorbate(s), Cremophor(s);
(7) a suitable buffer system, preferably citrate and/or phosphate sufficient to control pH from about 4.5 to about 7.0;
(8) a suitable thickener, preferably hydroxyethyl cellulose or polyvinyl pyrrolidone to build sufficient rheology of the formulation;
(9) an emollient, preferably glycerin in an amount from about 1 to about 30%.

### Example 12

### Confocal Imaging Studies Using Fluorescently Labeled DEX

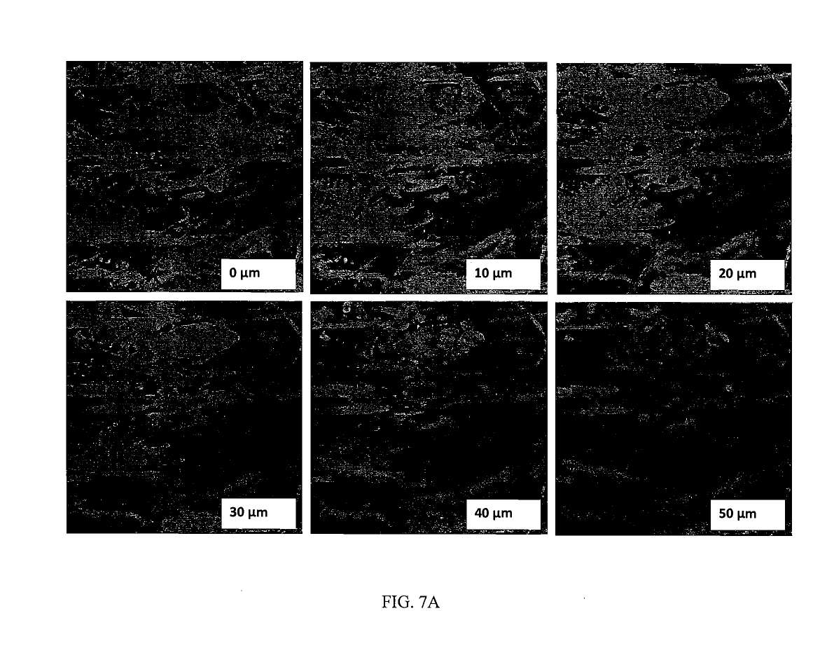

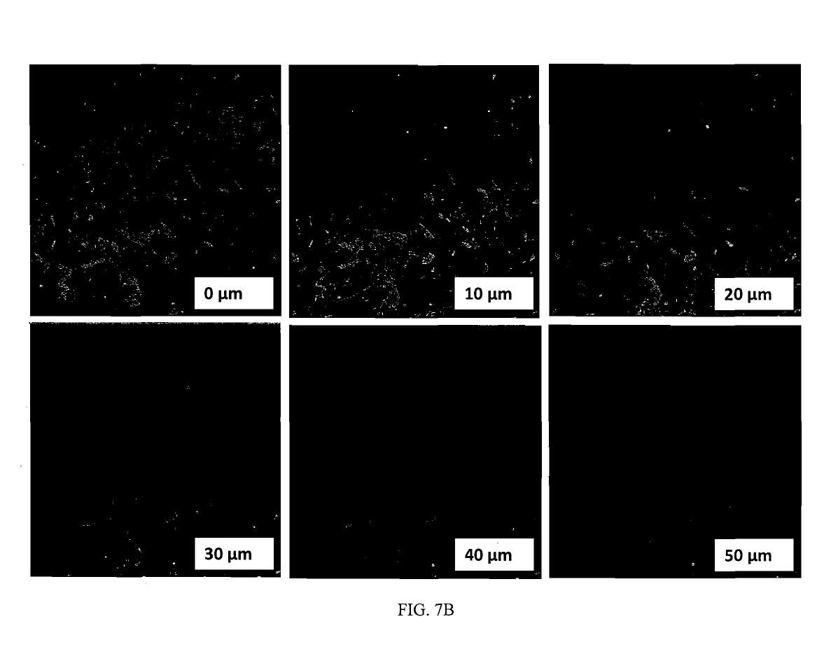

Confocal microscopy of hairless rat skin delivered iontophoretically (0.2 mA/cm² for 15 min, anodal ITP for electroosmosis) with 1 mg/mL DEX-fluorescein was performed up to 50 micrometer depth from the surface of the skin (FIGS. 7A and 7B). Following delivery in Franz diffusion cells, the surface of the skin was wiped with Kimwipe and further cleaned with a wet Kimwipe before imaging. Fluorescent intensity observed at 10 micrometer segments revealed that iontophoresis enhanced the amount and penetration depth of labeled DEX in the 0-50 micrometer range compared to passive delivery, as observed by higher fluorescent intensity observed along the depth of the skim Passively delivered DEX-fluorescein exhibited minor labeling on the surface of the skin only. These imaging studies further demonstrate the potential of iontophoresis to drive drugs into the skin.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A formulation suitable for iontophoretic delivery of a glucocorticoid comprising the glucocorticoid in an amount from about 0.01 to about 30% (w/w), a stabilizer and a buffer system sufficient to maintain the pH from about 4.0 and about 8.0.

2. The formulation of claim 1, further comprising an agent that has the ability to slow the release of the glucocorticoid from the epidermis to the dermis.

3. The formulation of claim 1, further comprising a preservative.

4. The formulation of claim 1, further comprising a thickening agent.

5. The formulation of claim 1, further comprising an emollient.

6. The formulation of claim 1, further comprising a solubilizing agent.

7. The formulation of claim 1, further comprising an alcohol.

8. The formulation of claim 1, wherein the glucocorticoid is selected from the group consisting of hydrocortisone, triamcinolone, betamethasone, dexamethasone, clobetasol, fluticasone, mometasone, fludroxycortide, fluocinonide, alclometasone, difluorocortolone and fluocinolone and a pharmaceutically active derivative thereof.

9. A formulation suitable for iontophoretic delivery of dexamethasone, or a pharmaceutically acceptable derivative thereof, comprising dexamethasone, or pharmaceutically acceptable derivative thereof, in an amount from about 1 to about 30% (w/w), a stabilizer and a buffer system sufficient to maintain the pH from about 5.0 and about 7.5.

10. The formulation of claim 9, wherein the formulation comprises dexamethasone sodium phosphate.

11. The formulation of claim 10, further comprising an agent that has the ability to slow release of dexamethasone from the epidermis to the dermis.

12. The formulation of claim 11, wherein the agent is selected from the group consisting of a saturated fatty acid, an unsaturated fatty acid, a glycol ether and a polyethylene glycol.

13. The formulation of claim 10, further comprising a preservative.

14. The formulation of claim 10, wherein the preservative is benzalkonium chloride.

15. The formulation of claim 10 further comprising a thickening agent.

16. The method of claim 15, wherein the thickening agent is selected from the group consisting of hydroxyethylcellulose and polyvinylpyrrolidone.

17. The formulation of claim 10, further comprising an emollient.

18. The method of claim 17, wherein the emollient is glycerin.

19. The formulation of claim 10, wherein the stabilizer is selected from the group consisting of an alcohol, a chelating agent and an antioxidant.

20. The formulation of claim 19, wherein the alcohol is selected from the group consisting of benzyl alcohol and ethanol.

21. The formulation of claim 19, wherein the chelating agent is disodium edetate.

22. The formulation of claim 19, wherein the antioxidant is selected from the group consisting of butylated hydroxyl anisole, butylated hydroxytoluene, creatine, sodium sulfite and methionine.

23. The formulation of claim 10 wherein the buffer system is selected from the group consisting of a citrate buffer, a phosphate buffer and a combination thereof.

24. A formulation suitable for iontophoretic delivery of triamcinolone or a pharmaceutically acceptable derivative thereof in an amount from about 0.01 and about 30%, a stabilizer, a solubilizing agent and a buffer system sufficient to maintain the pH from about 4.5 and 7.0.

25. The formulation of claim 24, wherein the formulation comprises triamcinolone acetonide.

26. The formulation of claim 25, wherein the stabilizer is selected from the group consisting of an alcohol, a chelating agent and an antioxidant.

27. The formulation of claim 26, wherein the alcohol is selected from the group consisting of benzyl alcohol and ethanol.

28. The formulation of claim 26 wherein the chelating agent is disodium edetate.

29. The formulation of claim 26, wherein the antioxidant is selected from the group consisting of butylated hydroxyl anisole, butylated hydroxytoluene, creatine, sodium sulfite and methionine.

30. The formulation of claim 25, further comprising an agent that has the ability to slow the release of triamcinonolone or a pharmaceutically acceptable derivative thereof from the epidermis to the dermis.

31. The formulation of claim 30, wherein the agent that has the ability to slow the release of triamcinolone or a pharmaceutically acceptable derivative thereof is selected from the group consisting of a saturated fatty acid and polyethylene glycol and a combination thereof.

32. The formulation of claim 25, further comprising a preservative.

33. The formulation of claim 32, wherein the preservative is benzalkonium chloride.

34. The formulation of claim 25, further comprising a thickening agent.

35. The formulation of claim 34, wherein the thickening agent is selected from the group consisting of hydroxyethyl cellulose and polyvinylpyrrolidone.

36. The formulation of claim 25, further comprising an emollient.

37. The formulation of claim 36, wherein the emollient is glycerin.

38. The formulation of claim 25, wherein the solubilizing agent is selected from the group consisting of polyethylene glycol, propylene glycol, polysorbate, Cremophor and combinations thereof.

39. The formulation of claim 25, wherein the buffer system is selected from the group consisting of a citrate buffer and a phosphate buffer.

40. A method of administering a glucocorticoid to a patient in need thereof, the method comprising iontophoretically delivering a formulation suitable for iontophoretic delivery to a body surface of said patient, said formulation comprising a glucocorticoid comprising the glucocorticoid in an amount from about 0.01 to about 30% (w/w), a stabilizer and a buffer system sufficient to maintain the pH from about 4.0 and about 8.0.

41. A method of administering dexamethasone, or a pharmaceutically acceptable derivative thereof, to a patient in need thereof, the method comprising iontophoretically delivering a formulation suitable for iontophoretic delivery of dexamethasone, or a pharmaceutically acceptable derivative thereof, to a body surface of said patient, said formulation comprising dexamethasone, or pharmaceutically acceptable derivative thereof, in an amount from about 1 to about 30% (w/w), a stabilizer and a buffer system sufficient to maintain the pH from about 5.0 and about 7.5.

42. A method of administering triamcinolone, or a pharmaceutically acceptable derivative thereof, to a patient in need thereof, the method comprising iontophoretically delivering a formulation suitable for iontophoretic delivery of triamcinolone, or a pharmaceutically acceptable derivative thereof, to a body surface of said patient, said formulation comprising triamcinolone in an amount from about 0.01 and about 30%, a stabilizer, a solubilizing agent and a buffer system sufficient to maintain the pH from about 4.5 and 7.0.

43. A method of treating an inflammatory condition in a patient in need thereof, the method comprising iontophoretically delivering a formulation suitable for iontophoretic delivery to a body surface of said patient, said formulation comprising a glucocorticoid comprising the glucocorticoid in an amount from about 0.01 to about 30% (w/w), a stabilizer and a buffer system sufficient to maintain the pH from about 4.0 and about 8.0.

44. A method of treating an inflammatory condition in a patient in need thereof, the method comprising iontophoretically delivering a formulation suitable for iontophoretic delivery of dexamethasone, or a pharmaceutically acceptable derivative thereof, to a body surface of said patient, said formulation comprising dexamethasone, or pharmaceutically acceptable derivative thereof, in an amount from about 1 to about 30% (w/w), a stabilizer and a buffer system sufficient to maintain the pH from about 5.0 and about 7.5.

45. A method of treating an inflammatory condition in a patient in need thereof, the method comprising iontophoretically delivering a formulation suitable for iontophoretic delivery of triamcinolone, or a pharmaceutically acceptable derivative thereof, to a body surface of said patient, said formulation comprising triamcinolone in an amount from about 0.01 and about 30%, a stabilizer, a solubilizing agent and a buffer system sufficient to maintain the pH from about 4.5 and 7.0.
